(19) United States
(12) Patent Application Publication
(10) Pub. No.: US 2010/0185278 A1
(43) Pub. Date: Jul. 22, 2010

(54) Apical Papillary Msucle Attachment for Left Ventricular Reduction

(75) Inventor: Kemal Schankereli, Stillwater, MN (US)

Correspondence Address: JUNEAU PARTNERS, 333 N Fairfax St., Suite 305, ALEXANDRIA, VA, 22301, US

(73) Assignee: TENDYNE MEDICAL

(21) Appl. No.: 12/691,591

(22) Filed: Jan. 21, 2010

Related U.S. Application Data
(60) Provisional application No. 61/146,144, filed on Jan. 21, 2009.

(51) Int. Cl. A61F 2/24

(52) U.S. Cl. 623/2.36

(57) ABSTRACT

This invention relates to devices and methods for the therapeutic changing of the geometry of the left ventricle of the human heart. Specifically, the invention relates to the apical introduction of an anchoring device to align the papillary muscles.

### CROSS REFERENCE TO RELATED APPLICATIONS

This application claims priority benefit under 35 USC 119(e) to U.S. 61/146,144 entitled Apical Papillary Muscle Attachment for Left Ventricular Reduction, filed 21 Jan. 2009, the contents of which are hereby incorporated in their entirety.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

n/a

### REFERENCE TO A SEQUENCE LISTING

A table or a computer list appendix on a compact disc
[ ] is
[X] is not
   included herein and the material on the disc, if any, is incorporated-by-reference herein.

### BACKGROUND

1. Field of the Invention

This invention relates to devices and methods for the therapeutic changing of the geometry of the left ventricle of the human heart. Specifically, the invention relates to the apical introduction of an anchoring device to align the papillary muscles.

2. Background of the Invention

According to the Center for Disease Control, heart disease is the leading cause of death in the United States and is a major cause of disability. Almost 700,000 people die of heart disease in the U.S. each year. That is about 29% of all U.S. deaths. Heart disease is a term that includes several more specific heart conditions.

One of these conditions is cardiomyopathy. Cardiomyopathy is a weakening of the heart muscle or a change in heart muscle structure. It often results in inadequate heart pumping or other heart function abnormalities. These can result from various causes, including prior heart attacks, viral or bacterial infections, and others.

The geometry of the myocardium is critical to proper functioning. The myocardium is comprised of a single, continuous tissue that wraps around itself, spiraling up from the apex of the heart, to form a helix with elliptically shaped ventricles. This spiral produces an oblique muscle fiber orientation, meaning that the fibers form a more ventricle ‘x’ shape, so that when fibers shorten 15%, it produces a 60% ejection fraction. Because of its elliptical shape and defined apex, the ventricle is subjected to a relatively low level of lateral stress.

However, a dilated left ventricle is generally due to the effects of a myocardial infarction. An occlusion, or blockage, of cardiac arteries results in either an akinetic (non-beating) or dyskinetic (irregular beating) tissue downstream from the occlusion. This downstream ventricular tissue is damaged, but since the volume of blood that fills the ventricle does not change, the damaged organ has to work harder to eject the blood. This increased load causes an increase in the radius of the ventricle and the thickness of the ventricular wall changes. Further, the apex of the heart becomes circular, the remaining myocardial tissue suffers from pathological hypertrophy, and the valve opening widens. As the ventricle dilates, the muscle fiber orientation, which is critical to a good ejection fraction, becomes transverse, or more horizontal. Subsequently, the ejection fraction decreases; a 15% shortening of muscle fibers now produces only a 30% ejection fraction. The lateral stress on the ventricle increases. Overall, the dilated left ventricle cannot produce a strong enough pulse to maintain health and efficient circulatory return.

Ventricular reduction is a well-known type of operation in cardiac surgery to reduce enlargement of the heart from cardiomyopathy. In 1985, Vincent Dor, MD, introduced endoventricular circular patch plasty (EVCPP), or the Dor procedure, as a viable method for restoring a dilated left ventricle to its normal, elliptical geometry. The Dor procedure, which uses a circular suture and a Dacron® patch to correct LV aneurysms and exclude scarred parts of the septum and ventricular wall, has been one option for ventricular remodeling. The procedure restores ventricular shape, increases ejection fraction, decreases the left ventricular end systolic volume index (LVESVI), and allows for complete coronary revascularization.

The disadvantage to the Dor procedure is that it places synthetic tissue inside the LV cavity and it is usually done as part of a coronary artery bypass graft (open heart) surgery.

Others have attempted further solutions to this problem. U.S. Pat. No. 7,060,021 to Wilk discloses a type clamp for the left ventricle which pulls opposing walls of the heart together in order to close off lower portions of both ventricles.

U.S. published patent application 2007/0083076 to Lichtenstein discloses methods and devices for altering the blood flow through the left ventricle by engaging the outer surface of the heart in a type of binding.

U.S. published patent application 2008/0293996 to Evans discloses a system and method for volume reduction by inserting a conical polymeric container, i.e. balloon, into the left ventricle to reduce the volume of blood flow.

Additionally, many patents and publications are directed to the catheter based repair of the mitral valve using various types of sutures and tethers. For example, U.S. published patent application 2008/0243150 to Starksen discloses a valve annulus treatment device secured by anchors that cinch or draw together circumferentially to tighten the valve annulus (ring). Starksen also discloses that such a device can be delivered by advancing a catheter through the aorta. Published PCT patent application WO/2006/135536 to De Marchena discloses a papillary muscle tether for left ventricular reduction by delivery either (1) through the femoral vein and delivered to the left ventricle via a trans-septal approach into the left atrium, across the mitral valve, or (2) retrograde through the femoral artery, advanced through the aortic valve, and into the left ventricle. However, cardiac catheterization poses the risk of blood clots that can trigger strokes, damage to blood vessels, and damage to the heart or pericardium. Thus, procedures and devices which address these and other concerns are needed in the field.

### BRIEF SUMMARY OF THE INVENTION

Accordingly, in a preferred embodiment of the invention, there is provided a method for improving cardiac function, comprising the steps of: inserting a tether device into a patient; and inserting said tether device through the apex of the patient's heart and into the left ventricle of the patient's heart; and attaching at least one first papillary muscle anchor of said tether device to a first papillary muscle within said left ventricle; and attaching at least one second papillary muscle anchor of said tether device to a second papillary muscle within the left ventricle wall of the patient's heart; and wherein said papillary anchor and said wall anchor are joined by a tether member so as to change the geometry and reduce the volume of the left ventricle.

In another preferred embodiment of the invention, there is provided a method for reducing ventricular volume, comprising the steps of: inserting a tether device into a patient; and inserting said tether device through the apex of the patient's heart and into the left ventricle of the patient's heart; and attaching at least one first papillary muscle anchor of said tether device to a first papillary muscle within said left ventricle; and attaching at least one second papillary muscle anchor of said tether device to a second papillary muscle of the left ventricle of the patient's heart; and wherein said papillary anchor and said wall anchor are joined by a tether member so as to reduce the volume of the left ventricle.

In another preferred embodiment of the invention, there is provided a method as described herein further comprising the step of adjusting the tether member to achieve a desired geometry of the left ventricle.

In another preferred embodiment of the invention, there is provided a method as described herein further comprising the steps of attaching at least one additional papillary anchor joined by an additional tether member so as to achieve a desired geometry of the left ventricle.

In another preferred embodiment of the invention, there is provided a method as described herein further comprising the step of adjusting the tether member to achieve coaptation of the mitral valve.

In another preferred embodiment of the invention, there is provided a method as described herein further comprising wherein the inserting of said tether device includes passing said tether device through a trocar sleeve or cannula

In another preferred embodiment of the invention, there is provided a method as described herein further comprising where inserting the tether device into a patient is performed by inserting a catheter into the patient through the vascular system of the patient.

In another preferred embodiment of the invention, there is provided a method as described herein further comprising implanting a hemostasis valve at the apex insertion site on the heart of the patient, wherein said valve is a blood leakage control valve/sleeve.

In another preferred embodiment of the invention, there is provided a medical device for improving cardiac function or reducing ventricular volume, comprising: a cannula having a tethering device disposed therein; said cannula having a trocar for piercing the apex of the patient's heart and a leakage control hemostasis valve/sleeve; said tethering device comprising at least one first papillary muscle anchor for attaching to a first papillary muscle within said left ventricle and at least one second papillary muscle anchor for attaching to the second papillary muscle of the left ventricle of the patient's heart; said tethering device further comprising a tether member for joining said first papillary muscle anchor to said second papillary muscle anchor so as to reduce the left ventricular volume of the patient.

In another preferred embodiment of the invention, there is provided a device as described herein wherein said tether member has an adjustable mechanism for adjusting the length of said tether.

In another preferred embodiment of the invention, there is provided a device as described herein further comprising at least one additional papillary anchor joined by an additional tether member so as to achieve a desired geometry of the left ventricle.

In another preferred embodiment of the invention, there is provided a device as described herein wherein the tether member is comprised of nitinol (nickel-titanium shape memory alloy) or austinetic stainless steel.

### BRIEF DESCRIPTION OF THE DRAWINGS

FIG. 1A is a graphical representation of an apical introduction device used to align papillary muscles. FIG. 1A shows cannula and the tethering member with four protruding anchors and depth gauge.

FIG. 1B is a graphical representation of an apical introduction device used to align papillary muscles. FIG. 1B shows cannula and the tethering member with three protruding anchors and depth gauge.

FIG. 1C is a graphical representation of an apical introduction device used to align papillary muscles. FIG. 1C shows cannula and the tethering member with one protruding anchor and depth gauge.

FIG. 2 is a drawing of a heart having an enlarged left ventricle.

FIG. 3 is a drawing of a heart being apically pierced by a catheter.

FIG. 4 is a drawing of a heart having a first tether implanted in a papillary muscle.

FIG. 5 is a drawing of a heart having a second tether implanted in an opposing papillary muscle.

FIG. 6 is a drawing of a heart showing two papillary muscles tethered.

FIG. 7 is a drawing of a heart showing the tethers being cinched.

FIG. 8 is a drawing of corrected heart showing the tethers gathered by an adjustable connector.

FIG. 9 is a drawing of a heart showing a circular tether embodiment.

FIG. 10 is a photo representation of a heart in cross-section being pierced by a device and shows inserting at the apex.

FIG. 11 is a photo representation of a heart in cross-section being pierced through the papillary muscle.

FIG. 12 is a photo representation of a heart in cross-section being pierced by a device at the apex, and shows interaction with a papillary muscle.

FIG. 13 is a photo representation of a heart in longitudinal cross-section.

FIG. 14 is a photo representation of a heart in cross-section showing attachment of tether lines (in blue), prior to being cinched, or joined.

### DETAILED DESCRIPTION OF THE INVENTION

### Definitions

The following definitions are provided as an aid to understanding the detailed description of the present invention.

“Anchors” for the purposes of this application, is defined to mean any fastener. Thus, anchors may comprise C-shaped or semicircular hooks, curved hooks of other shapes, straight hooks, barbed hooks, clips of any kind, T-tags, or any other suitable fastener(s). In one embodiment, anchors may comprise two tips that curve in opposite directions upon deployment, forming two intersecting semi-circles, circles, ovals, helices or the like. In some embodiments, anchors are self-deforming. By “self-deforming” it is meant that anchors change from a first undeployed shape to a second deployed shape upon release of anchors from restraint in housing. Such self-deforming anchors may change shape as they are released from housing and enter papillary or myocardial tissue, to secure themselves to the tissue. Thus, a crimping device or other similar mechanism is not required on distal end to apply force to anchors to attach them to tissue.

Self-deforming anchors may be made of any suitable material, such as a super-elastic or shape-memory material like Nitinol or spring stainless steel. In other embodiments, anchors may be made of a non-shape-memory material and made be loaded into housing in such a way that they change shape upon release. Alternatively, anchors that are not self-deforming may be used, and such anchors may be secured to tissue via crimping, firing or the like. Even self-securing anchors may be crimped in some embodiments, to provide enhanced attachment to tissue. In some embodiments, anchors may comprise one or more bioactive agent. In another embodiment, anchors may comprise electrodes. Such electrodes, for example, may sense various parameters, such as but not limited to impedance, temperature and electrical signals. In other embodiments, such electrodes may be used to supply energy to tissue at ablation or sub-ablation amounts. Delivery of anchors may be accomplished by any suitable device and technique, such as by simply releasing the anchors. Any number, size and shape of anchors may be included in housing.

Apical or apex refers to a known part of the heart, roughly equivalent to the peak at the bottom of the organ.

Canula or cannula refers to a well-known tube-like medical instrument. It can be fitted with a trocar, a sharp pointed device for piercing tissue.

Tether may be one long piece of material or two or more pieces and may comprise any suitable material, such as Nitinol, austinetic steel, suture, suture-like material, a Dacron strip or the like.

Hemostasis valve, or valve/sleeve, refers to a device which allows the heart tissue to be pierced at the apex region with little or no blood loss. Similar valves/sleeves are well known in the venipuncture field where individual vacutainers can be repeatedly mounted on a single needle, and valves such as the Touehy Borst valve which allows multiple insertions of catheters while maintaining hemostasis.

Generally, delivery of the tether device may be advanced by any suitable advancing or device placement method so long as it arrives at the apex of the heart. Many catheter-based, minimally invasive devices and methods for performing intravascular procedures, for example, are well known, and any such devices and methods, as well as any other devices or method later developed, may be used to advance or position delivery device into a desired location. For example, in one embodiment a steerable guide catheter is first advanced percutaneously to the apex region. The steerable catheter is inserted into the left ventricle of the heart through the apex of the heart and thus into the space formed by left ventricle. An obturator pushes or holds the tissue in place once it has been pierced. Once in this space, the steerable catheter is easily advanced to the papillary muscle or to the ventricular wall, the anchor may then be advanced and inserted into the papillary muscle and/or the LV myocardium. Of course, this is but one exemplary method and any other suitable method, combination of devices, etc. may be used.

Referring now to the FIGUREs:

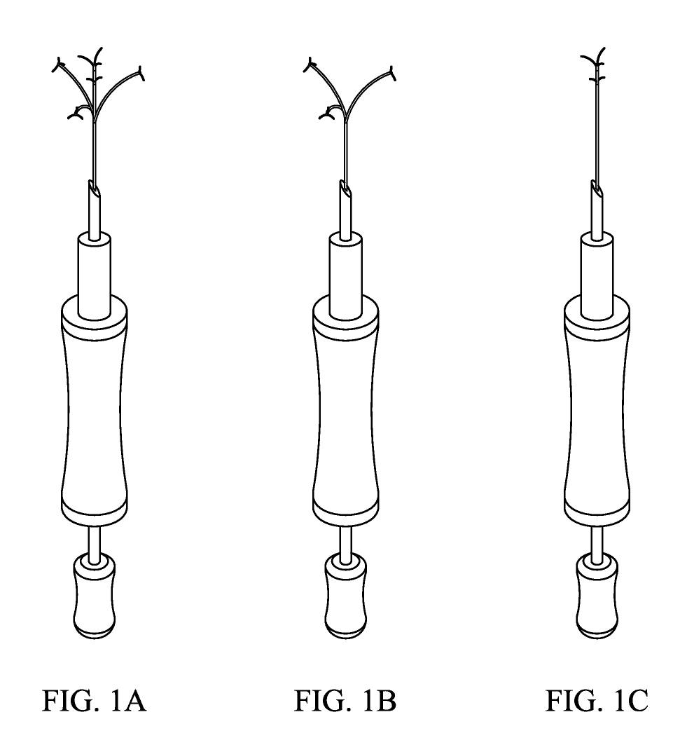

FIG. 1A is a graphical representation of an apical introduction device used to align papillary muscles. FIG. 1A shows cannula and the tethering member with four protruding anchors and depth gauge.

FIG. 1B is a graphical representation of an apical introduction device used to align papillary muscles. FIG. 1B shows cannula and the tethering member with three protruding anchors and depth gauge.

FIG. 1C is a graphical representation of an apical introduction device used to align papillary muscles. FIG. 1C shows cannula and the tethering member with two protruding anchors.

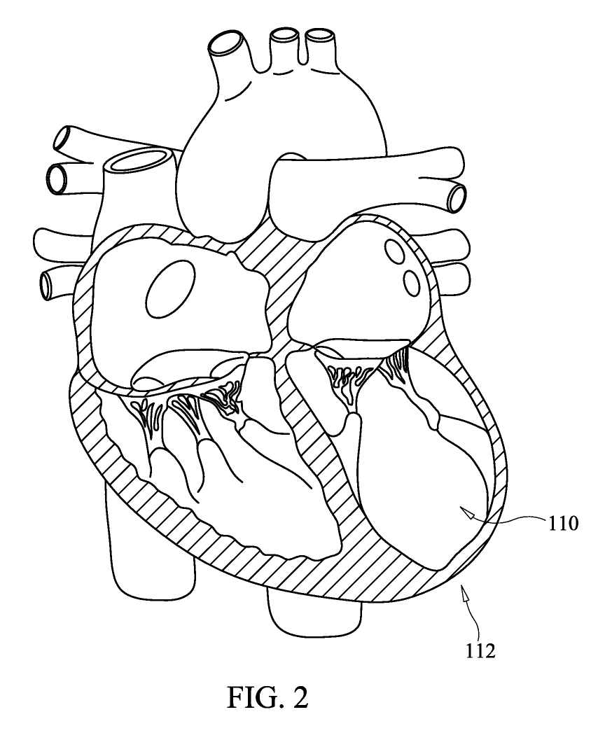

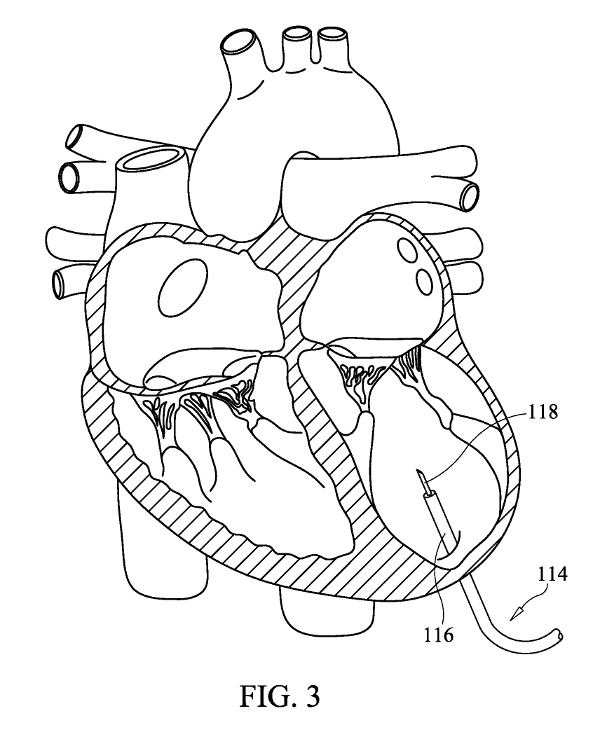

FIGS. 2-8 show a heart having an enlarged left ventricle 110, and the instant apical approach 112 to the left ventricle 110 is depicted in FIGS. 3-8. In this example embodiment, FIG. 3 shows the left ventricle is accessed by inserting a catheter 114 having a cannula 116 and trocar 118 that is advanced from into the left ventricle 110. Once the catheter 114 reaches the interior of the left ventricle, the trocar 118 is removed in favor of a steerable guide catheter 120 which permits introduction of the instruments which will be used to engage and tether the papillary muscles, as described in more detail below.

An advantage of the apical approach is that it eliminates any risks associated with crossing the aortic valve, trans-septal puncture, or arterial damage, and permits the use of larger French catheter, and provides direct access to the papillary muscles, without requiring that the mitral valve be crossed.

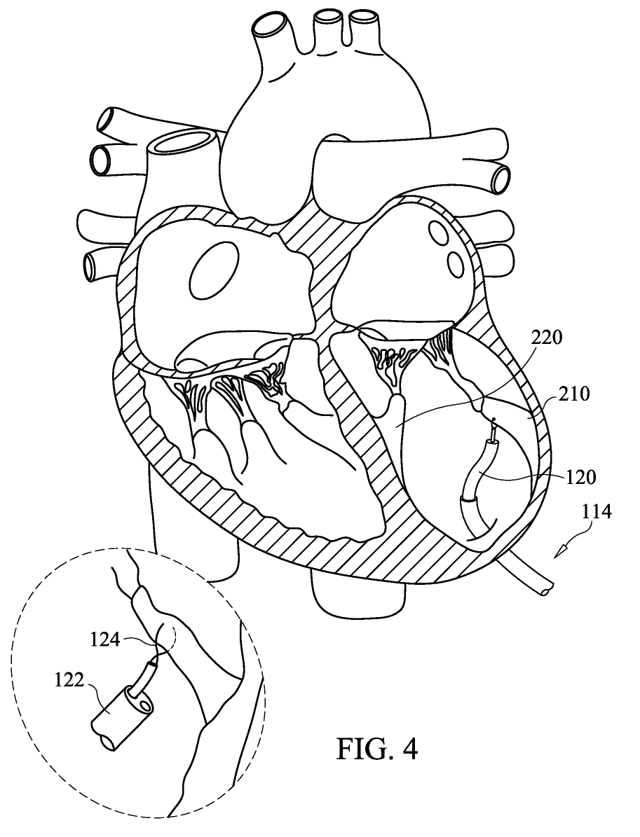

Referring now to FIGS. 4 and 4A, the papillary muscles 210, and 220 need to be address using the proper orientation of the catheters, tools and the like throughout the procedure. Such orientation is accomplished using a steerable catheter 120 or equivalent tool.

In an example embodiment of the invention, the papillary muscles 210, 220 are grasped by partial or full penetration or piercing. This may be accomplished with a variety of grasping mechanisms, preferably including one or more piercing prongs extending from an instrument or catheter tool so as to grasp a target structure. Referring more specifically to the example embodiment of FIG. 4, steerable catheter 120 is fed through the guide catheter 114 to secure a first anchor 124 of a tether structure 122 (see inset FIG. 3A) to one of the papillary muscles 210 in the left ventricle.

The steerable catheter 120 is advanced from the distal end of the guide catheter 114 and may be observed in real time via any conventional imaging technique. In the illustrated example embodiment, a suture or clip applying instrument (tethering device) 122 is passed through the catheter 120. Advantageously, the instrument has a steerable tip so that it may be directed to a position in opposed facing relation to a target portion of a papillary muscle. Disposed at or adjacent the distal end of the tethering instrument 122 in this embodiment is a clamp or clip 124 for secure attachment to the respective papillary muscle. The clip or clamp is advanced out of the deployment catheter and into engagement with respective papillary muscle. Any suitable mechanism can be sued to close the clip. If deemed necessary or desirable, one or more additional clips with tethers may be applied.

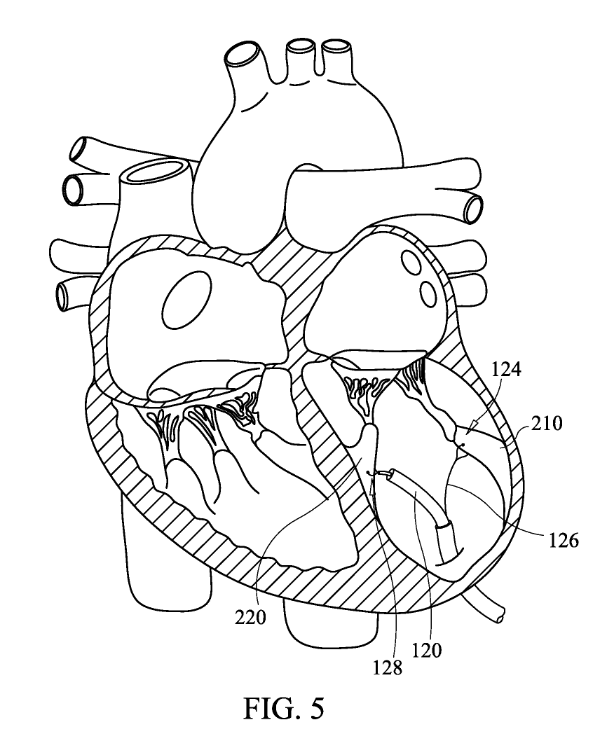

Referring now to FIG. 5, once the clip has been secured with respect to a first one of the papillary muscles 210, the instrument is withdrawn to reveal the flexible strand and the same or another instrument carrying another clip is conducted through the guide catheter adjacent the already placed flexible strand. In the alternative, the instrument carries at least first and second clips and respective flexible strands so that the papillary muscles can be respectively engaged without withdrawing the instrument and reinserting it. Whether the clips are attached sequentially by the sequential feed of an instrument or sequentially by manipulating the instrument, after each papillary muscle has been engaged by respective clip(s) with respective flexible strand(s), the instrument is withdrawn through the guide catheter.

According to an alternate embodiment, non-absorbable suture loop(s) may be applied directly in the papillary muscles. For example, a variation of the Perclose A-T® vasculature closure device, which is a stitch knot transmitting device with a suture cutter could be used apply a suture loop. There are also known laparoscopic devices, such as the Quik-Stitch Endoscopic Suturing System, that may be adapted to transvascularly securing a tether to the papillary muscles.

As illustrated in FIG. 5, the guide catheter 120 remains in place with the flexible tether strand(s) 126 extending therethrough from the respective secured clip/anchor 124 on first papillary muscle 210. Then, steerable catheter 120 attaches second anchor 128 to second papillary muscle 220.

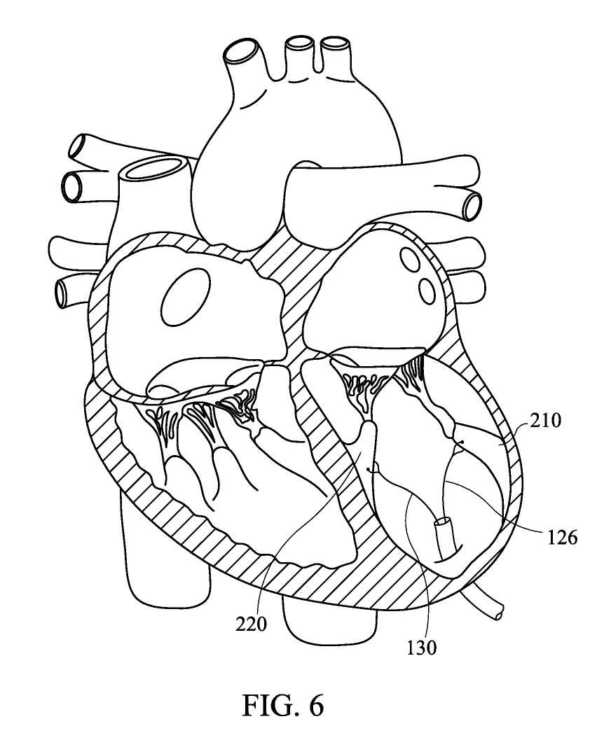

Referring now to FIG. 6, the tethered papillary muscles 210, 220 are tethered by tether strand 126 and 130.

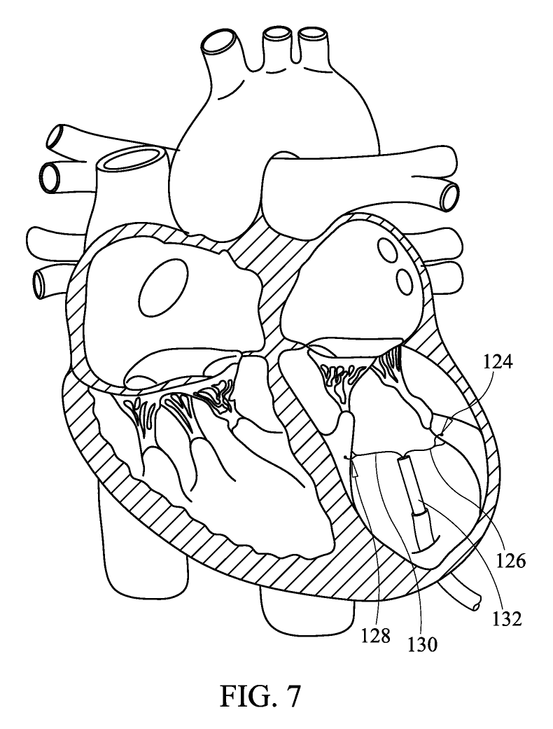

Referring now to FIG. 7, the tether strands 126 and 130 are next drawn together by using a gathering instrument 132, which is advanced over the flexible tethers and the tethers are pulled through the instrument to draw the clips 124, 128 toward one another. The tethers are then either tied or fastened together to define the desired spacing of the papillary muscles. For example, two tethers may have a knot transmitted to define the junction, or they are clipped to one another through the existing guiding catheter.

The tethering and drawing of the papillary muscles towards one another may be conducted while monitoring the position of the muscles fluoroscopically, and under intra-cardiac ultrasound guidance, so that the papillary muscles can be drawn to a desired transventricular distance. Intra cardiac Echo Doppler can also be used to assess the severity of LV enlargement/CV disease, or regurgitation, to adjust the length of the tethers to an optimum transventricular distance to suppress cardiac deficiency or regurgitation. So bringing the papillary muscles closer together reduces the size of the left ventricular cavity and will limit further distension of the ventricular wall, thereby mimicking the effect of the congenital false tendon to improve ventricular geometry and mitigate the effects of Dilated Cardiomyopathy.

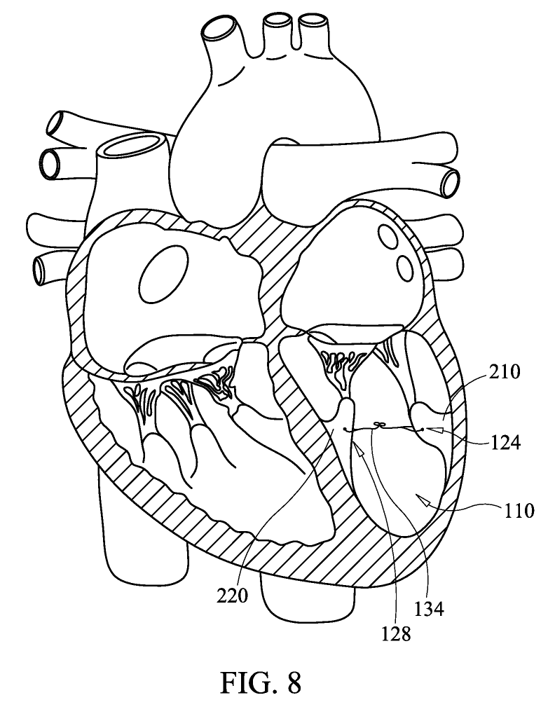

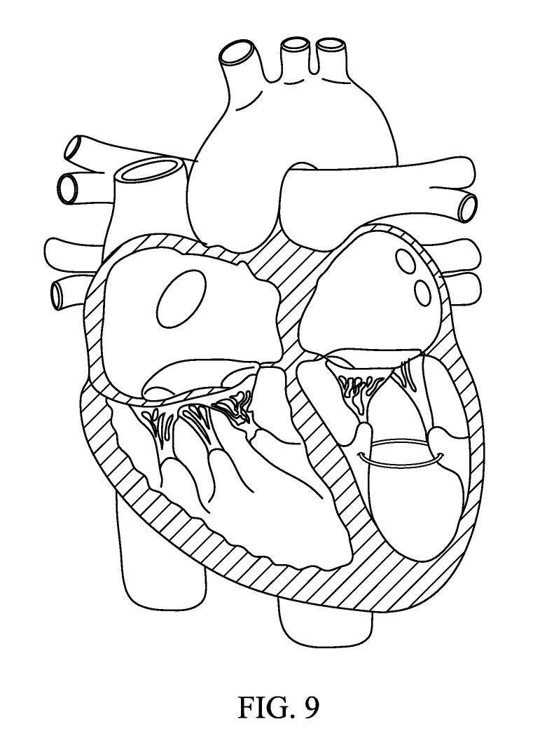

FIGS. 8 and 9 show corrected left ventricle 110 having papillary 210 held by anchor 124, and papillary 220 held by anchor 128, and joined by connector 134, which may be adjustable. Any suitable instrument may be used to capture and sever the excess tether length such as, for example, a suture trimmer.

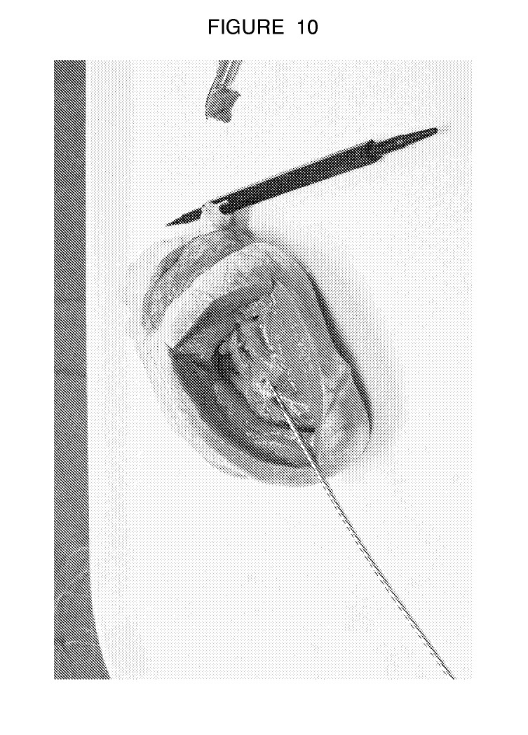

FIG. 10 is a photo representation of a heart in cross-section being pierced by a device and shows inserting at the apex.

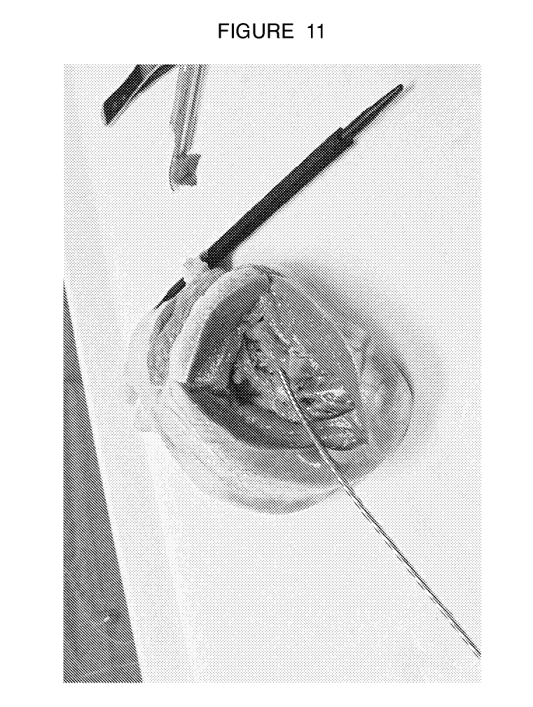

FIG. 11 is a photo representation of a heart in cross-section being pierced through the ventricular wall.

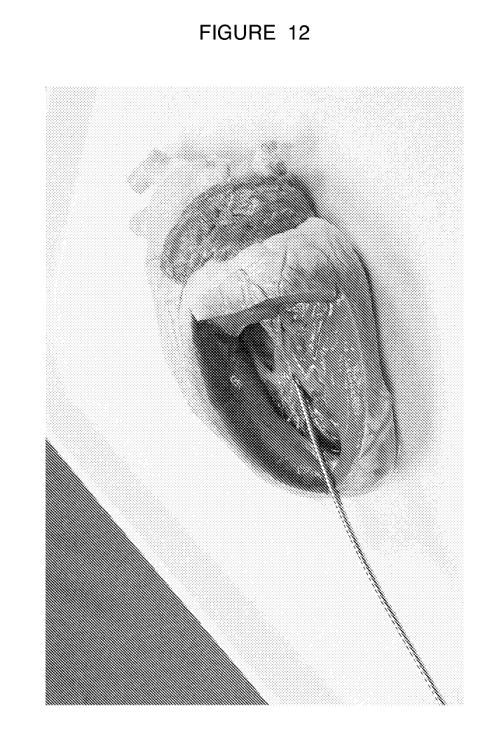

FIG. 12 is a photo representation of a heart in cross-section being pierced by a device at the apex, and shows interaction with a papillary muscle.

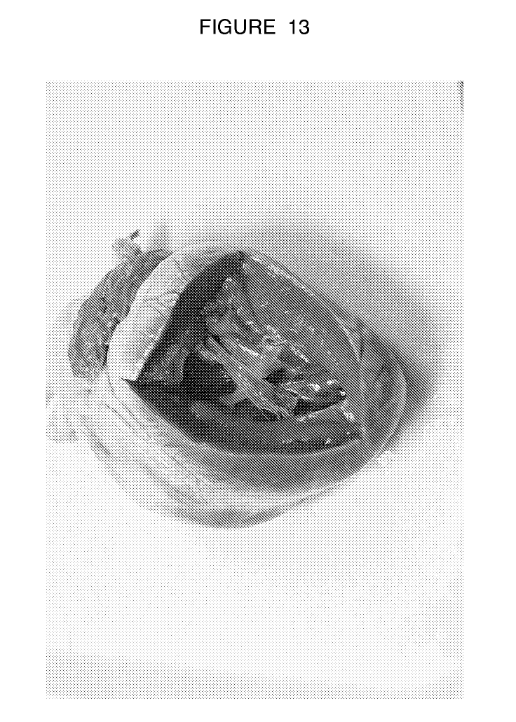

FIG. 13 is a photo representation of a heart in cross-section.

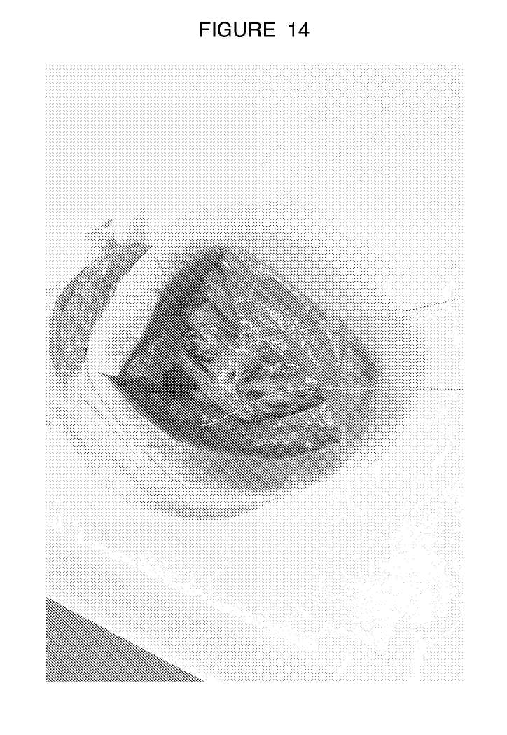

FIG. 14 is a photo representation of a heart in cross-section showing attachment of tether lines (in blue), prior to being cinched, or joined.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A method for improving cardiac function, comprising the steps of:
inserting a tether device into a patient; and
inserting said tether device through the apex of the patient's heart and into the left ventricle of the patient's heart; and
attaching a first papillary muscle anchor of said tether device to a first papillary muscle within said left ventricle; and
attaching a second papillary muscle anchor of said tether device to a second papillary muscle of the patient's heart; and
wherein said first papillary muscle anchor and said second papillary muscle anchor are joined by a tether member so as to reduce the volume of the left ventricle.

2. The method as claimed in claim 1, further comprising the step of adjusting the tether member to achieve a desired geometry of the left ventricle.

3. The method as claimed in claim 1, further comprising the steps of attaching at least one additional papillary muscle anchor joined by an additional tether member so as to achieve a desired geometry of the left ventricle.

4. The method as claimed in claim 1, further comprising the step of adjusting the tether member to achieve coaptation of the mitral valve. (Original)

5. The method as claimed in claim 1, further comprising wherein the inserting of said tether device includes passing said tether device through a trocar sleeve or cannula.

6. A method for reducing ventricular volume, comprising the steps of:
inserting a tether device into a patient; and
inserting said tether device through the apex of the patient's heart and into the left ventricle of the patient's heart; and
attaching a first papillary muscle anchor of said tether device to a first papillary muscle within said left ventricle; and
attaching a second papillary muscle anchor of said tether device to a second papillary muscle of the patient's heart; and
wherein said first papillary muscle anchor and said second papillary muscle anchor are joined by a tether member so as to reduce the volume of the left ventricle.

7. The method as claimed in claim 6, further comprising the step of adjusting the tether member to achieve a desired geometry of the left ventricle.

8. The method as claimed in claim 6, further comprising the steps of attaching at least one additional papillary muscle anchor joined by an additional tether member so as to achieve a desired geometry of the left ventricle.

9. The method as claimed in claim 6, further comprising the step of adjusting the tether member to achieve coaptation of the mitral valve.

10. The method as claimed in claim 6, further comprising wherein the inserting of said tether device includes passing said tether device through a trocar sleeve or cannula.

11. The method of claim 1 or 6, further comprising where inserting the tether device into a patient is performed by inserting a catheter into the patient through the vascular system of the patient.

12. The method of claim 1 or 6, further comprising implanting a valve at the apex insertion site on the heart of the patient, wherein said valve is a blood leakage control valve/sleeve.

13. A medical device for improving cardiac function or reducing ventricular volume, comprising: a cannula having a tethering device disposed therein; said cannula having a trocar for piercing the apex of the patient's heart and a leakage control valve/sleeve; said tethering device comprising at least one first papillary muscle anchor for attaching to a first papillary muscle within said left ventricle and at least one second papillary muscle anchor for attaching to a second papillary muscle of the patient's heart; said tethering device further comprising a tether member for joining said first papillary muscle anchor to said second papillary muscle anchor so as to reduce the left ventricular volume of the patient.

14. The device of claim 13, wherein said tether member has an adjustable mechanism for adjusting the length of said tether.

15. The device of claim 14, further comprising at least one additional papillary muscle anchor joined by an additional tether member so as to achieve a desired geometry of the left ventricle.

16. The device of claim 13, wherein the tether member is comprised of nitinol (nickel-titanium shape memory alloy) or austinetic stainless steel.
